(19) United States
(12) Patent Application Publication
(10) Pub. No.: US 2015/0273215 A1
(43) Pub. Date: Oct. 1, 2015

(54) SYSTEMS AND METHODS FOR ASSESSMENT OF PAIN AND OTHER PARAMETERS DURING TRIAL NEUROSTIMULATION

(71) Applicant: PACESETTER, INC., Sylmar, CA (US)

(72) Inventors: Yelena Nabutovsky, Mountain View, CA (US); Gene A. Bornzin, Simi Valley, CA (US); Brad Maruca, Fairview, TX (US)

(73) Assignee: PACESETTER, INC., Sylmar, CA (US)

(21) Appl. No.: 14/226,567

(22) Filed: Mar. 26, 2014

(51) Int. Cl. A61N 1/36; A61N 1/05; A61N 1/372

(52) U.S. Cl. CPC A61N 1/36139; A61N 1/37252; A61N 1/05

(57) ABSTRACT

Techniques are provided for use with a trial neurostimulation device having a lead for implant within a patient. In one example, neurostimulation is delivered using the lead while an indication of patient pain is detected. Various functions of the trial device are then controlled in response to patient pain, such as by adjusting neurostimulation control parameters to improve pain reduction, recording diagnostic information representative of patient pain or transmitting such parameters to a separate external instrument for analysis. In this manner, patient pain is automatically detected to provide objective feedback as to the efficacy of trial neurostimulation. Various embodiments of flexible trial neurostimulation device patches are described herein, including patches that are adhesively mounted over the point of entry of the trial lead into the patient, thus providing a comfortable patch that hygienically isolates the point of entry of the lead.

### FIELD OF THE INVENTION

The disclosure generally relates to implantable neurostimulation devices and, in particular, to trial neurostimulation devices for use with implantable leads.

### BACKGROUND OF THE INVENTION

Implantable neurostimulation devices can be employed to manage pain arising from a variety of neuropathies and is a valuable treatment for chronic intractable neuropathic pain. Neurostimulation is also being investigated for cardiac applications such as treatment of heart failure and atrial fibrillation. To these various ends, a spinal cord stimulation (SCS) device or other neurostimulator may be implanted within the body to deliver electrical pulses to nerves or other tissues. The neurostimulator typically includes a small pulse generator device similar to a pacemaker but equipped to send electrical pulses to leads mounted along the nerves near the spinal cord or elsewhere within the body. For SCS, the generator is often implanted in the abdomen. The stimulation leads may include thin wires or paddles for delivering electrical pulses to patient nerve tissues. An external controller, similar to a remote control device, may be provided to allow the patient to control or adjust the neurostimulation. Currently, prior to permanent (i.e. chronic) implant of a neurostimulator, the patient undergoes a trial period during which he or she is implanted with a percutaneous lead that is externalized and connected to a trial neurostimulation control device or instrument, which the patient carries with him or her.

In United States, patients typically have the trial neurostimulation system for less than a week. In Europe, the trial period can last up to a month. During the trial period, the patient carries the neurostimulation system with him or her. Unfortunately, current trial neurostimulation devices are problematic. The implanted percutaneous lead can be inadvertently pulled from the epidural space or may migrate from the implant site such that the patient will not receive any therapeutic benefit. This can result in a failed trial. In addition, the current system is quite cumbersome. Typically, the lead is taped to the skin at the exit point. A long extension cord connects the lead to the trial neurostimulator, which is worn on a belt. The extension cord and lead are packaged within a bulky bandage and tape arrangement that is uncomfortable and irritating for the patient. With such devices, the patient is not allowed to shower. The trial experience can often be very unpleasant for patients. It is believed that the “annoyance factor” can lead to a failed trial because the patients become “fed up” with the process. As a result, many patients who might benefit from SCS or other forms of neurostimulation do not receive such devices, or the devices are programmed with inappropriate or ineffective parameters. Moreover, the only feedback typically provided regarding therapy effectiveness and optimal stimulation parameters is the subjective feedback given by the patient based on reported sensations.

Accordingly, it would be desirable to provide improved trial neurostimulation devices and it is to this end that aspects of the disclosure are generally directed.

### SUMMARY OF THE INVENTION

In an exemplary embodiment, a method is provided for use with a trial neurostimulation device having a neurostimulation lead for implant within a patient. With the method, neurostimulation is selectively delivered to the patient using the lead. An indication of patient pain is detected using the trial neurostimulation device and one or more functions of the trial neurostimulation device are then controlled in response to the indication of patient pain, such as adjusting neurostimulation control parameters, recording diagnostic information representative of patient pain or transmitting such parameters to a separate external instrument or programmer device. Hence, patient pain is detected by the trial device to provide objective feedback as to the efficacy of the trial neurostimulation. The neurostimulation may include SCS.

In an illustrative embodiment, a galvanic skin response (GSR) sensor is employed to detect an indication of patient pain and measure or quantify its intensity. Briefly, GSR is an electrodermal response during which there are changes in the electrical properties of the skin due, e.g., to a change in the psychological state of the patient. If a weak current or voltage is delivered to the skin, conductance can be measured indicative of GSR. Although there are normal fluctuations in GSR, an increase in the number of spikes in the signal can be indicative of pain. In one example, the device detects and counts spikes in a GSR signal and associates changes in the number of spikes with changes in the intensity of patient pain. In another example, the device evaluates the frequency content of the GSR signal using a Fast Fourier Transform (FFT) or similar technique and then associates changes in the frequency content of the GSR signal with changes in the intensity of the pain. An increase in the number of spikes or an increase in high frequency components of the GSR signal generally indicates an increase in pain, at least in the absence of confounding factors. To help discriminate changes in the GSR signal due to pain from changes due to confounding factors, the trial device preferably includes an activity sensor, a heart rate (HR) sensor and a blood pressure (BP) sensor. Since an increase in patient activity can increase GSR, the device separately detects and tracks patient pain during periods of activity and periods of relative inactivity. Still further, increases in HR and BP can be used to corroborate pain detection. In one example, if GSR increases but HR and BP do not increase, then the increase in GSR is not deemed to be indicative of an increase in patient pain.

Various device functions can be activated, deactivated, adjusted or otherwise controlled based on indications of patient pain. For example, pain metrics derived from GSR can be selectively stored within a device memory and/or transmitted to an external diagnostic instrument for clinician review, along with corresponding HR values, BP values and activity values. These metrics may be used to objectively determine the efficacy of the pain relief therapy and can be used during clinical trials. The metrics may also be used for optimization of pulse stimulation waveforms, frequency and intensity, as well as to adjust a percentage of time and the time of day over which therapy is delivered. Other parameters that can be controlled in response to patient pain include pulse polarity and parameters for controlling burst pacing. Still further, the trial device can be equipped to distinguish between an initial baseline evaluation interval and a subsequent trial stimulation interval. That is, methods are provided for measuring and interpreting information related to patient status before and during a trial period. In one such example, the device begins its operation within a baseline evaluation interval during which it detects patient pain and records diagnostic data without neurostimulation. Indeed, in some examples, neurostimulation components such as the pulse generator may not even be deployed during this interval, just the sensing components. Following the baseline interval, neurostimulation is then provided to the patient while continuing to monitor pain to determine the efficacy of neurostimulation and to adjust or optimize the neurostimulation control parameters in a feedback loop to reduce or minimize pain. Values obtained during the baseline period can be compared to values obtained during the trial period to provide an objective assessment of whether the patient responds to neurostimulation therapy. Additionally or alternatively, therapy may be automatically controlled during a clinical trial to determine whether stimulation “on” or “off” yields different pain metrics. This can be especially useful in connection with burst stimulation because such stimulation is not accompanied by paresthesia. In examples described herein, the neurostimulation is primarily SCS but the systems and methods described herein can be applied to other forms of neurostimulation as well.

In another exemplary embodiment, a neurostimulation patch device is provided for use with an implantable neurostimulation lead for implant within a patient. The neurostimulation patch device includes: a body member having a bottom portion adapted to be detachably affixed to patient skin, typically over the implant site of the implantable lead; a neurostimulation circuit located within the body member and configured to output neurostimulation signals; and a connector located within the body member and configured to electrically couple the neurostimulation circuit to the implantable lead, wherein the bottom portion of the body member defines an opening for passage of an end of the implantable lead for connection to the connector. The patch device further includes one or more sensors operative to sense physiological signals. A pain detection system can be provided that detects an indication of patient pain based on signals received from the sensors. The sensors may include a GSR sensor for detecting an indication of patient pain, as well as an electrocardiogram (ECG) sensor for detecting HR, a pulse oximeter for detecting BP and an activity sensor such as an accelerometer for detecting the activity state of the patient. With the exemplary neurostimulation patch, patient pain can be conveniently detected and assessed while neurostimulation is selectively controlled. Depending upon the size, shape and adhesive properties of the patch, patient discomfort can be greatly reduced or eliminated compared to bulky predecessor trial devices. In an illustrative example, the trial patch is a unitary element with a built-in stimulator and a bandage that covers a percutaneous implant site. Excess lead may be coiled in a bandage cavity. The lead plugs directly into a connector in the bandage cavity. The trial patch is taped to the skin of the patient and is typically not visible under patient clothing. The patient can shower because the patch seals around the implant site. The lead is also protected from pulling and dislodgement. The trial patch can greatly improve the overall trial experience for the patient, leading to fewer failed trials.

System and method examples are described in detail below.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 illustrates an exemplary trial SCS patch device equipped for pain detection and configured to be adhesively attached to the patient;

FIG. 2 provides an overview of techniques for pain assessment for use by the trial SCS patch device of FIG. 1 or similarly-equipped trial medical devices;

FIG. 3 provides an exemplary procedure in accordance with the general method of FIG. 2 wherein GSR is employed to assess patient pain and wherein an initial baseline pain evaluation period is employed;

FIG. 4 further illustrates exemplary techniques for assessing pain based on GSR use with the procedure of FIG. 3;

FIG. 5 provides a set of pain assessment procedures wherein, in some examples, patient activity, HR and BP are also measured for use with the procedure of FIG. 3;

FIG. 6 is a block diagram illustrating pertinent components of the trial SCS patch device of FIG. 1;

FIG. 7 is a schematic illustration of an exemplary trial SCS patch corresponding generally to the device of FIG. 1;

FIG. 8 is a simplified diagram of an embodiment of the stimulation patch of FIG. 7 that physically connects to a lead;

FIG. 9 provides a top “outer side” planar view of an exemplary trial SCS patch embodiment generally corresponding to the device of FIG. 8, shown without the stimulation lead;

FIG. 10 provides a bottom “inner side” planar view of the exemplary trial SCS patch of FIG. 9, shown with the stimulation lead;

FIG. 11 provides a top “outer side” planar view of another exemplary trial SCS patch embodiment generally corresponding to the device of FIG. 8, shown without the stimulation lead; and

FIG. 12 provides a bottom “inner side” planar view of the exemplary trial SCS patch of FIG. 10, shown with the stimulation lead.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators are used to refer to like parts or elements throughout.

Overview of Trial Neurostimulation System with Pain Assessment

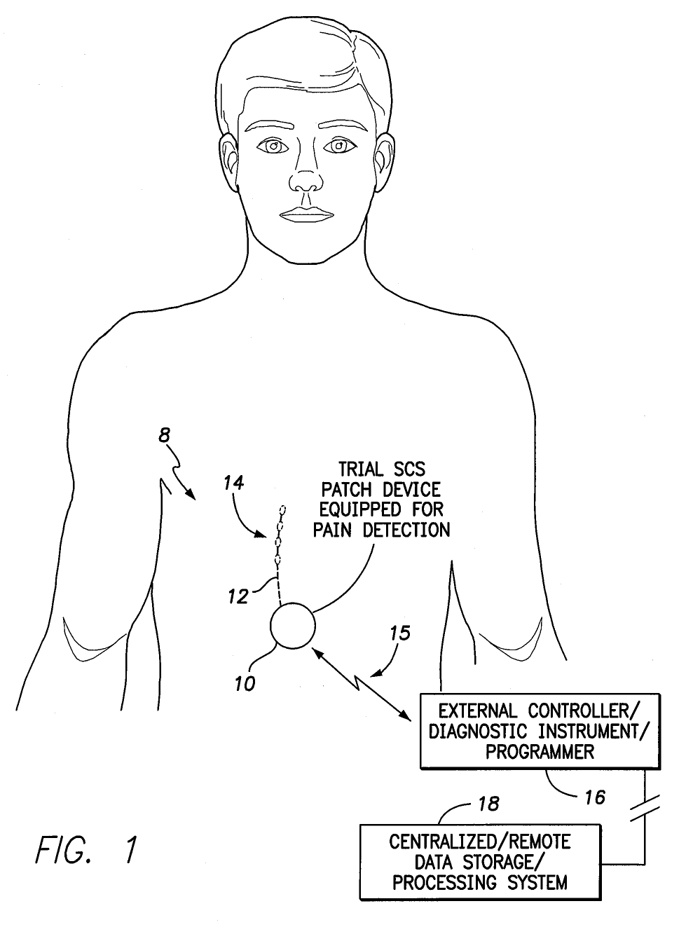

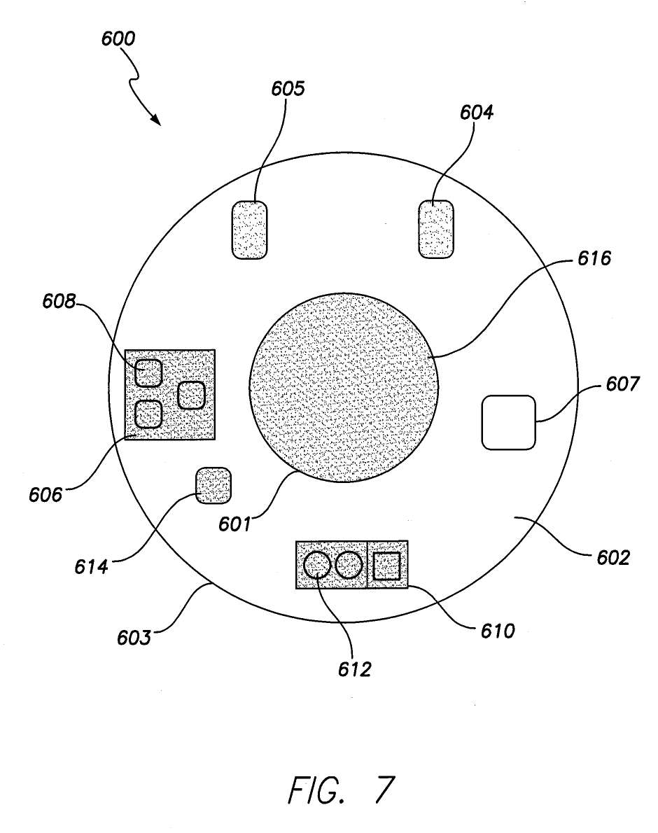

FIG. 1 illustrates an exemplary trial medical system 8 having an external trial SCS neurostimulation patch device 10 equipped to deliver neurostimulation to a patient on which the device is affixed and also equipped to assess, track or evaluate patient pain using one or more sensors (not specifically shown in FIG. 1.) Trial SCS device 10 employs, in this example, a percutaneous lead 12 with a set of electrodes 14 implanted within the patient for delivering the trial neurostimulation to patient nerve tissues. In the drawing, phantom lines are used to illustrate the implanted portion of lead 12 whereas solid lines illustrate external patch device 10 so as to distinguish the components implanted within the body from those kept external to the patient. Although not specifically shown in FIG. 1, a proximal end of lead 12 is connected into a bottom, inner or “skin side” portion of patch device 10 via an opening in the patient skin so as to allow the pulse generator and other electronics of the SCS device to be externalized from the patient whereas the electrodes along the distal end of the lead are internalized within the tissues of the patient. With this configuration, the point of entry of the lead into the patient can be hygienically sealed under the patch. Further details regarding physical embodiments of the patch device are provided below with reference to FIGS. 7-12.

Typically, the electrodes of a trial SCS lead such as percutaneous lead 12 are positioned near suitable nerves of the spinal column to allow for efficacious pain reduction via neurostimulation. However, in other examples, the electrodes might be placed elsewhere within the patient. Moreover, it should be understood that the percutaneous lead of FIG. 1 is merely exemplary. Four electrodes are shown in the example, although more or fewer electrodes can be employed. For example, the device might employ an eight-electrode Octrode™ lead, which is a type of linear eight electrode percutaneous lead provided by St. Jude Medical. Still further, in other examples, paddle electrode leads or other lead shapes or configurations can be used. Typically, the lead is removed upon completion of the trial period and replaced with a new lead if implantation of a permanent (i.e. chronic or long-term) SCS system is warranted. However, in some examples, the stimulation lead can be retained within the body, with the external device disconnected from the lead and replaced with a fully implantable neurostimulation controller that is then coupled to the implanted lead. See, for example, techniques described in U.S. patent application Ser. No. 13/940,727 of Nabutovsky et al., filed Jul. 12, 2013, entitled “Fully Implantable Trial Neurostimulation System Configured for Minimally-Intrusive Implant/Explant” (Atty. Docket A13P3007.)

In the example of FIG. 1, trial patch SCS device 10 is equipped to communicate with an external controller/diagnostic instrument/programmer 16 using radio-frequency (RF) or other wireless signals to transmit data collected by the trial device (including data pertaining to patient pain) and/or to receive commands from the external instrument to activate, deactivate or adjust neurostimulation. The commands may specify various stimulation sets (Stim Sets) initially specified by a clinician. The Stim Sets specify SCS parameters for controlling delivery of SCS to nerve tissues of the patient to address the needs of the patient, such as to reduce pain or to achieve desired cardioprotective effects. The clinician or the patient can then change the Stim Sets using external instrument 16 via a wireless communication link 15 such as to change the amplitude, frequency or duration of stimulation pulses generated by the SCS device. The communication link may employ Bluetooth or other suitable wireless communication protocols. In some examples, the external instrument is a suitably-equipped tablet computer or smartphone, which may be referred to as a “Neuro External” device. See, for example, U.S. patent application Ser. No. 14/012,634 of Wu et al., filed Aug. 28, 2013, entitled “Systems and Methods for Low Energy Wake-Up and Pairing for use with Implantable Medical Devices” (Atty Docket A13P3012). External instrument 16 may also be equipped to communicate with a centralized/remote data processing system 18 via the Internet or other suitable communication channels/networks to relay information to the primary care physician of the patient or to other appropriate clinicians. The centralized system may include or employ such systems as the HouseCall™ remote monitoring system or the Merlin@home/Merlin.Net systems of St. Jude Medical.

Although the example of FIG. 1 shows a trial device 10 for stimulating the spinal cord, additional or alternative stimulation devices might be employed, such as devices for stimulating other tissues or organs within the patient. Some patients might additionally have an implantable cardiac rhythm management device (CRMD) such as a pacemaker, implantable cardioverter-defibrillator (ICD) or a cardiac resynchronization therapy device (CRT), which is not shown in the figure. Note also that FIG. 1 is a stylized illustration that does not necessarily set forth the precise locations of the various device components nor their relative sizes or shapes.

### Exemplary Pain Assessment Systems and Methods

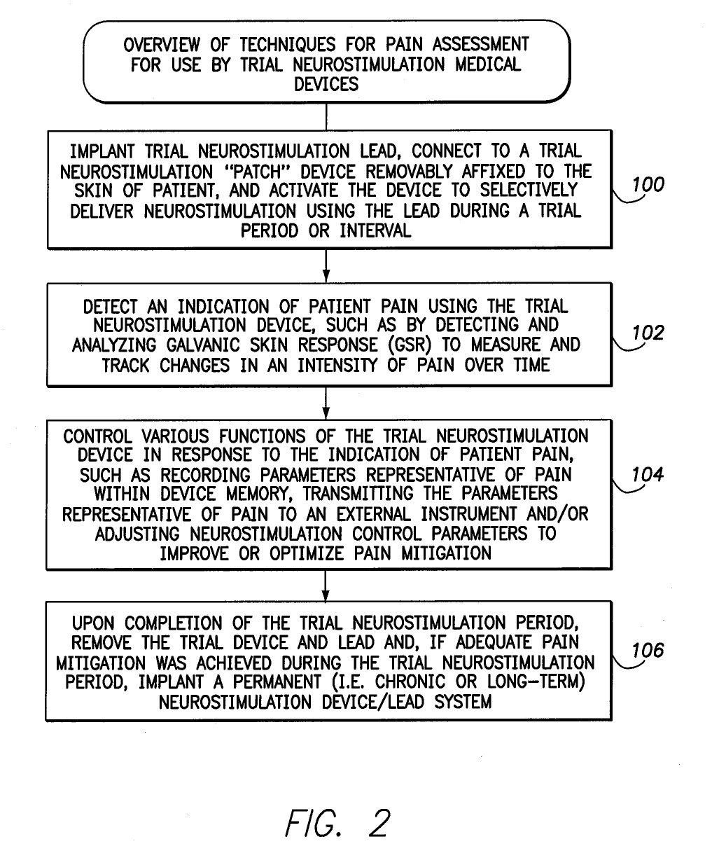

FIG. 2 broadly summarizes techniques for pain assessment for use by trial neurostimulation medical devices such as the trial SCS device of FIG. 1. Although advantageously employed within the patch device configuration of FIG. 1, these pain assessment techniques can be implemented within other trial neurostimulation devices that do not necessarily employ a patch configuration or within suitable non-neurostimulation devices such as medical devices directed to other forms of therapy. Beginning at step 100, a suitable trial stimulation lead is implanted within a patient and connected (either physically or wirelessly) to a trial neurostimulation “patch” device, which is removably affixed to the skin of the patient. The device is then activated to selectively deliver neurostimulation therapy using the lead during a trial neurostimulation period. At step 102, an indication of patient pain is detected using the trial neurostimulation device, such as by detecting and analyzing GSR using a suitable sensor so as to measure and track changes in an intensity of pain over time. At step 104, one or more functions of the trial neurostimulation device are controlled in response to the indication of patient pain, such as by recording parameters representative of pain within device memory, transmitting the parameters representative of pain to an external instrument and/or adjusting neurostimulation control parameters to improve or optimize pain mitigation. At step 106, upon completion of the trial neurostimulation period, the trial device and lead are removed and, if adequate pain mitigation was achieved during the trial neurostimulation period, a permanent (i.e. chronic or long-term) neurostimulation device/lead system is implanted. As already noted, in some examples, the lead itself need not be removed but is merely coupled to the long-term, implantable SCS device.

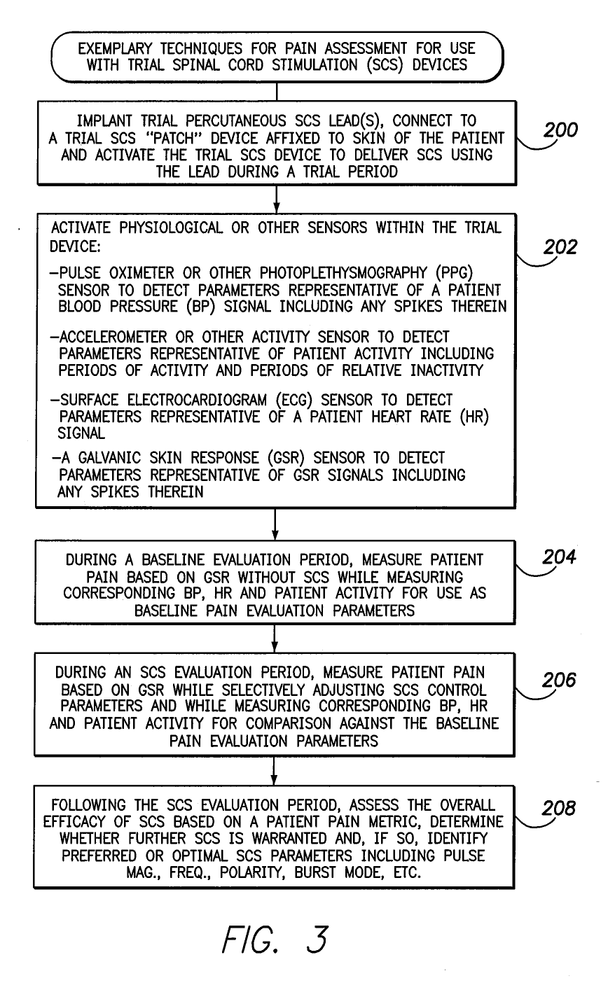

FIG. 3 provides further information regarding exemplary techniques for pain assessment particularly for use by a trial SCS device employing a percutaneous lead where either the device or the lead are equipped with sensors to detect BP, HR, patient activity and GSR. In the examples described herein, the trial patch device is equipped with suitable physiological or other sensors. In other implementations, the lead could instead include one or more of sensors for detecting at least some of these parameters for relaying to the patch device for processing therein. Also, it should be understood that two or more leads could instead be employed. The exemplary procedure FIG. 3 employs an initial trial baseline evaluation period for collecting data without neurostimulation for comparison against data subsequently collected during a trial SCS evaluation period. In other examples, particularly where the overall trial period is intended to be relatively short, the trial period need not be split into separate intervals. It is noted, though, that by providing a comfortable trial patch system to replace cumbersome conventional trial systems, patients will likely be far more willing to wear the trial device for longer intervals of time, thus allowing plenty of time to collect ample baseline data without SCS and then collecting additional data during neurostimulation for comparison and evaluation.

Beginning at step 200, one or more trial percutaneous SCS leads are implanted and connected to a trial SCS patch device affixed to skin of the patient. The SCS device is activated to deliver SCS using the lead during a trial period. At step 202, the following sensors are activated within the trial device: a pulse oximeter or other photoplethysmography (PPG) sensor to detect parameters representative of a patient BP signal including any spikes or changes therein; an accelerometer or other activity sensor to detect parameters representative of patient activity including periods of activity and periods of relative inactivity; a surface ECG sensor to detect parameters representative of a patient HR signal; and a GSR sensor to detect parameters representative of GSR signals including any spikes or changes therein.

Techniques for assessing pain via GSR are discussed, for example, in U.S. Pat. No. 8,512,240 to Zuckerman-Stark et al. See, also, Storm, “Changes in Skin Conductance as a Tool to Monitor Nociceptive Stimulation and Pain,” Current Opinion in Anesthesiology, 2008; 21:296-804. Pulse oximeters are discussed, for example, in U.S. Patent Application 2009/0187087 of Turcott, “Analysis of Metabolic Gases by an Implantable Cardiac Device for the Assessment of Cardiac Output.” Techniques for assessing BP based, at least in part, on surface ECGs are described in U.S. Pat. No. 8,162,841 to Keel et al., entitled “Standalone Systemic Arterial Blood Pressure Monitoring Device.” Accelerometers and activity monitors are discussed, for example, in U.S. Pat. No. 7,177,684 to Kroll et al., entitled “Activity Monitor and Six-minute Walk Test for Depression and CHF Patients.” Surface ECG detection techniques are discussed, for example, in U.S. Pat. No. 7,136,703 to Cappa et al., entitled “Programmer and Surface ECG System with Wireless Communication.”

At step 204, during the initial baseline evaluation period, the trial device measures patient pain based on GSR without SCS while measuring and storing corresponding BP, HR and patient activity values for use as baseline pain evaluation parameters. At step 206, during a subsequent SCS evaluation period, the trial device measures patient pain based on GSR while selectively adjusting SCS control parameters and while measuring and storing corresponding BP, HR and patient activity for comparison against the baseline pain evaluation parameters. In one particular example, the baseline period might last a few days or a week while the subsequent SCS evaluation period might last two or three weeks, allowing ample data to be collected, yet without any significant annoyance or inconvenience to the patient since the trial device is configured as a patch. At step 208, following the SCS evaluation period, the trial device (or an external instrument equipped to receive data from the trial device) analyzes GSR and other collected data to assess the overall efficacy of the trial SCS based, e.g., on a patient pain metric that quantifies patient pain. That is, the trial device may calculate a pain metric intended to provide an objective assessment of patient pain that can be used in conjunction with any subjective indications of pain provided by the patient to the clinician. Also at step 208, the trial device, an external instrument or the supervising clinician then determines whether further SCS is warranted based on patient pain data and, if further SCS is warranted, preferred or optimal SCS parameters are identified including particular Stim Sets and/or particular values for pulse magnitude, pulse frequency, pulse polarity, as well as any applicable burst mode parameters, etc. Burst patterns for neurostimulation are discussed, for example, in U.S. Patent Application 2009/0299435 of Gliner et al., entitled “Systems and Methods for Enhancing or Affecting Neural Stimulation Efficiency and/or Efficacy.”

It should be understood that any “optimal” SCS parameters identified using these techniques are not necessarily absolutely optimal in any rigorous mathematical sense. As can be appreciated, what constitutes optimal depends on the criteria used for judging the resulting performance, which can be subjective in the minds of patients and clinicians. Accordingly, the SCS parameters identified herein are at least “preferred” parameters. Clinicians and/or patients may choose to adjust or alter the SCS parameters via device programming at their discretion.

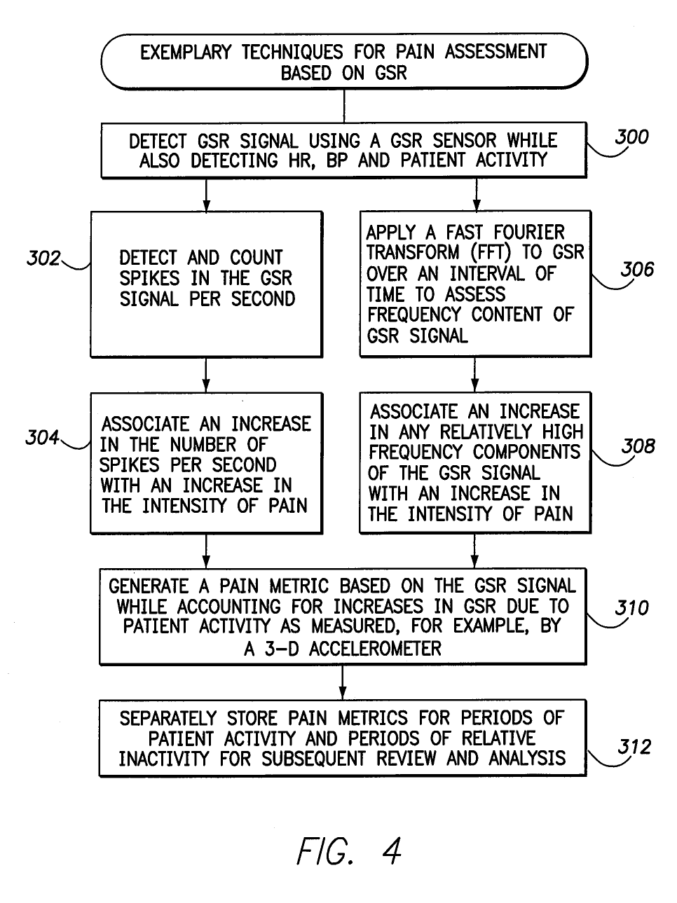

Turning now to FIG. 4, exemplary techniques for assessing and quantifying pain based on GSR will now be described. Beginning at step 300, the trial device detects GSR signals using a GSR sensor while also detecting HR, BP and patient activity, using suitable sensors as already discussed. At steps 302 and 304, the trial device detects and counts spikes in the GSR signal occurring per second (or within any other suitable interval of time) and then associates an increase in the number of spikes with an increase in the intensity of pain. Again, see the above-cited paper by Storm et al., particularly FIG. 2 therein, which shows spikes within a GSR signal. GSR spikes can be detected at step 302 by, for example, using otherwise conventional signal detection techniques based on the magnitude and rate of change of the signal for comparison against applicable thresholds. The count of spikes per second can thereby provide an objective and quantified pain metric, whereby an increase in the number of spikes indicates an increase in patient pain, and vice versa. Various thresholds or other parameters employed for spike-based pain quantification may be specified by, for example, determining the number of spikes per second within GSR signals measured in test patients in circumstances where the amount of pain is known.

Additionally or alternatively, at steps 306 and 308, the trial device applies an FFT (or similar) to the GSR signal collected over an interval of time (such as over the latest minute) to assess the frequency content of the GSR signal and then associates an increase in any relatively high frequency components of the GSR signal with an increase in the intensity of pain. In this regard, a frequency threshold may be specified and the presence of any significant spectral components of the GSR signal above that frequency is then deemed to be indicative of patient pain. Various thresholds or other parameters employed for FFT-based pain quantification may be specified by, for example, determining the spectral components of GSR signals measured in test patients in circumstances where the amount of pain is known.

At step 310, the trial device (or an external instrument receiving data from the trial device) generates a pain metric based on the GSR signal while accounting for increases in GSR due to patient activity as measured, for example, by a 3-D accelerometer. The pain metric may be based on either the spike-based pain assessment, the FFT-based pain assessment or a numerical combination of both. Techniques for generating a combined metric based on various parameters for evaluation are discussed, e.g., in: U.S. Pat. No. 7,207,947 to Koh et al. Insofar as patient activity is concerned, it is expected that increases in activity will cause a general increase in GSR and hence the trial device preferably analyzes GSR data collected during periods of relative inactivity separately from GSR data collected during periods of relative activity. A suitable activity threshold can be pre-determined to distinguish “activity” from “inactivity” based, e.g., on the magnitude of the output of an accelerometer-based activity sensor. At step 312, the trial device (or external instrument) separately stores pain metrics for periods of patient activity and periods of relative inactivity for subsequent review and analysis.

In this regard, general patient activity should cause an increase in the baseline GSR due to sweating. If the activity is associated with pain, the GSR should also exhibit an increase in the higher frequency component or the spikes per second, as already discussed. There may also be an increase in BP. The trial device preferably stores the amount of time that the patient is experiencing pain (as detected via GSR) and increased BP during activity. If activity sensor shows lack of movement, then HR, BP, and GSR should remain relatively stable. If during inactivity, HR increases, the number of spikes per second increases in the GSR, and BP increases, the trial device thereby determines the patient is feeling pain even without activity. The device then stores the amount of time the patient is experiencing higher spikes per second, elevated BP, and increased HR without activity in device memory. The two measurements—pain with activity and pain without activity—thereby provide an indication of whether the trial system is effective or not and provide feedback indicating which settings are associated with increased pain or decreased pain.

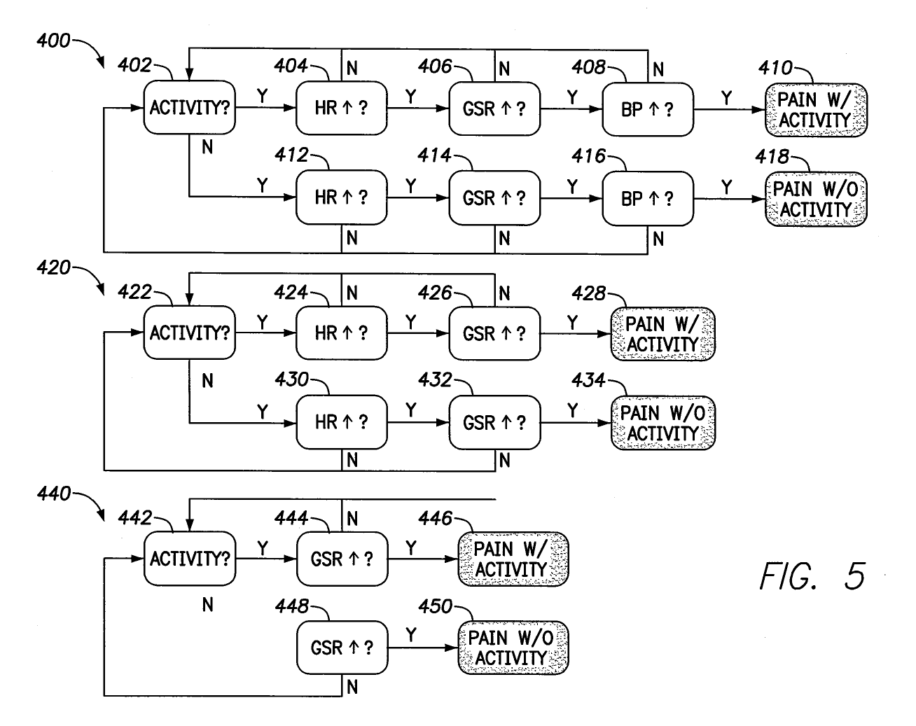

FIG. 5 schematically illustrates various procedures that may be used, depending upon the available sensors. Beginning with a first procedure 400, in which HR and BP sensors are included within the trial device, along with GSR and activity sensors, the trial device assesses patient activity at step 402. If patient activity exceeds a threshold indicative of “activity,” HR, GSR and BP are then assessed at steps 404, 406 and 408, respectively, for comparison against corresponding pre-determined activity baseline values, i.e. baseline values for HR, GSR and BP obtained during periods of patient activity. If each of these parameters exhibits a significant increase over their corresponding activity baseline values—including an increase in GSR spikes indicative of pain—then “pain with activity” is thereby indicated at step 410. If any of the sensors do not show a significant increase relative to their corresponding activity baseline values, then pain is not indicated. Conversely, if patient activity remains below a threshold indicative of “activity,” HR, GSR and BP are then assessed at steps 412, 414 and 416, respectively, for comparison against corresponding pre-determined inactivity baseline values, i.e. baseline values for HR, GSR and BP obtained during periods of patient inactivity. If each of these parameters exhibits a significant increase over corresponding pre-determined inactivity baseline values, then “pain without activity” is thereby indicated at step 418. If any of the sensors do not show a significant increase relative to their inactivity baseline values, then pain is again not indicated. As can be appreciated, the various activity baseline values are generally higher than corresponding inactivity baseline values, since patient activity tends to increase HR, BP and GSR, even in the absence of pain.

A second procedure 420 may be employed if there is no BP sensor. The trial device assesses patient activity at step 422 and, if the patient is active, HR and GSR are then assessed at steps 424 and 426, respectively, for comparison against corresponding activity baseline values. If both of these parameters exhibit a significant increase over corresponding baseline values, then “pain with activity” is indicated at step 428. If either sensor parameter does not show a significant increase relative to their corresponding baseline value, then pain is not indicated. Conversely, if the patient is inactive, HR and GSR are assessed at steps 430 and 432, respectively, for comparison against corresponding inactivity baseline values. If both of these parameters exhibit a significant increase over baseline values, then “pain without activity” is indicated at step 434. If either of the sensor does not show a significant increase relative to their inactivity baseline value, pain is again not indicated.

A third procedure 440 may be employed if there are no BP and HR sensors. The trial device assesses patient activity at step 442 and, if the patient is active, GSR is assessed at step 444 for comparison against a corresponding GSR activity baseline value. If a number of spikes in the GSR signal exhibit a significant increase over its corresponding activity baseline value, then “pain with activity” is indicated at step 446. Otherwise, pain is not indicated. Conversely, if the patient is inactive, GSR is assessed at step 448 for comparison against its corresponding activity baseline value. If the number of spikes in GSR exhibits a significant increase over its inactivity baseline value, then “pain without activity” is indicated at step 450. Otherwise, pain is not indicated.

To summarize some of the foregoing methods, in the presence of an accelerometer, HR sensor, BP sensor and GSR sensor, the following can be implemented. Activity is detected using the accelerometer and HR. If the accelerometer shows movement and HR is increased, the patient is deemed active. As noted, general activity should cause an increase in the baseline GSR due to sweating. If the activity is associated with pain, GSR should show an increase in the higher frequency components or spikes per second. There may also be an increase in BP. The amount of time that the patient is experiencing higher spikes per second in the GSR and increased BP during activity is stored. Periods of inactivity are detected using the accelerometer. If the accelerometer shows a lack of movement, HR, BP, and GSR should remain stable. If during inactivity, HR increases, the number of spikes per second increases in the GSR, and BP increases, the patient is deemed to be feeling pain even without activity. The amount of time the patient is experiencing higher spikes per second, elevated BP and increased HR without activity is stored. These two measurements—pain with activity and pain without activity—provide evidence that the trial system is effective or not and provide feedback indicating which settings are associated with increased pain or decreased pain. As shown, these general procedures can be performed without BP and/or HR measurements. Activity is then detected by the accelerometer alone and pain is judged by the GSR alone. Alternatively, if an activity sensor is not available either, the device can simply monitor spikes per second from the GSR and record periods of time when the rate of spikes per second has increased. An overall increase in spikes per second can be an indication of more pain. This information may be presented as a daily average or a histogram.

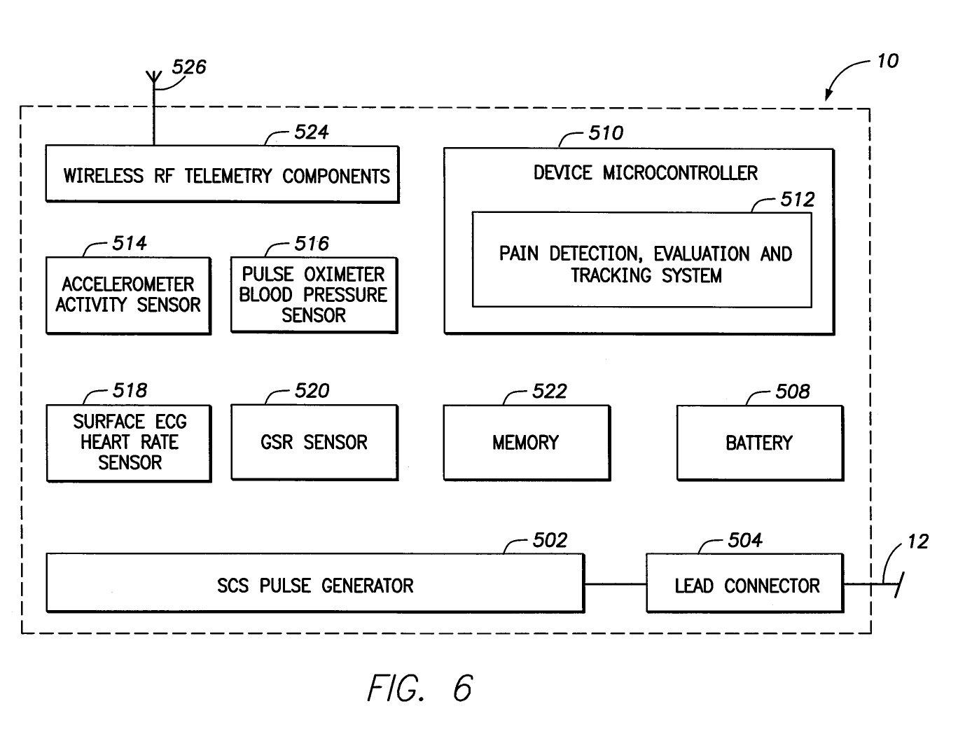

FIG. 6 provides a block diagram illustrating pertinent components of the trial patch device of FIG. 1 for use in delivering neurostimulation and implementing the pain assessment procedures of FIGS. 2-5. Briefly, in this example, trial device 10 includes an SCS pulse generator 502 coupled via a lead connector 504 to stimulation lead 12. The pulse generator and other active components of the trial device receive power from one or more batteries 508 and operate under the control of device microcontroller 510. With the exception of the connection between the pulse generator and the lead connector, connection lines are not shown. The microcontroller includes a pain evaluation and tracking system 512, which is operative to detect, quantify and track patient pain based on data collected from: an accelerometer activity sensor 514; a pulse oximeter blood pressure sensor 516; surface ECG heart rate sensor 518; and a GSR sensor 520; wherein pain assessment exploits the techniques described above. Data is stored in device memory 522 and/or transmitted to an external instrument via wireless RF telemetry components 524 using an antenna 526. Typically, the wireless RF telemetry components are also equipped to receive signals from the external instrument via the antenna, such as SCS programming commands. As can be appreciated, various other components may be included within the patch device to allow it to perform its intended functions, such as a device bus for relaying data and other signals among various components. Depending upon the implementation, the various components of the microcontroller may be implemented as separate software modules or the modules may be combined to permit a single module to perform multiple functions. The microcontroller, or some or all of the components, may be implemented using any suitable technology such as application specific integrated circuits (ASICs) or the like. Note that the various components of FIG. 6 are shown enclosed in a phantom line block to indicate that the components need not all be installed within a single hard device housing. In a typical implementation, the microcontroller, its memory and the SCS pulse generator might be enclosed within a single metallic device housing, with the various other components of the trial device mounted elsewhere within a flexible patch structure or apparatus.

For further information regarding neurostimulation systems and techniques, see, e.g.: U.S. patent application Ser. No. 13/442,749 of Xi et al., filed Apr. 9, 2012, entitled “Systems and Methods for Controlling Spinal Cord Stimulation to Improve Stimulation Efficacy for Use by Implantable Medical Devices” (Atty Docket A12P3005); U.S. Patent Application 2013/0325083 of Bharmi et al., entitled “Systems and Methods for Controlling Neurostimulation based on Regional Cardiac Performance for use by Implantable Medical Devices”; and U.S. Patent Application 2010/0331921 to Bornzin et al., entitled “Neurostimulation Device and Methods for Controlling Same.” See, also, techniques discussed in: U.S. Pat. No. 8,600,500 to Rosenberg et al., entitled “Method and System to Provide Neural Stimulation Therapy to Assist Anti-Tachycardia Pacing Therapy.”

### Exemplary Trial SCS Patch Embodiments

FIG. 7 is an illustration, partially in schematic form, of pertinent components of an exemplary trial SCS patch 600 that may be used as the trial SCS device of FIG. 1. The device is illustrated without the percutaneous SCS lead attached thereto. Patch 600 includes a patch body or assembly 602 that is generally and substantially circular, within which various components are mounted for positioning against the skin of the patient around a point of entry of the percutaneous SCS lead, i.e. above the implant site of the lead. The part of the patch containing the electronics may be referred to as the inner circle portion 601 and the outside part that contains the medical adhesive may be referred to as the outer circle portion or peripheral portion 603. In the example of FIG. 7, the sensors are all located on or within the outer circle portion so that the sensors can be placed on the patient prior to providing the patient with the electronics of SCS trial system. That is, the outer circle portion may be affixed to the patient without also providing the inner circle portion.

A sensor controller 607 may be provided within the outer circle portion, which is separate from the microcontroller of the inner circle components (not shown in FIG. 7) so as to accommodate embodiments where the outer circle components are separate from the inner circle components. Alternatively, the SCS controller of the inner circle portion may also control the sensors. In some examples, the sensor controller is physically wired to each sensor. In other examples, wireless interconnections may be employed. Batteries can be separately provided with each sensor or may be connected to the sensor controller or provided within the inner circle portion. If feedback from the sensors is to be used to automatically adjust therapy, the sensors can be connected via suitable connectors to the electronics of the inner circle portion. Alternatively, if so equipped, the sensors can directly and wirelessly transmit information to a separate programmer instrument. A common antenna for wireless transmission can be looped in or around the outer circle portion for connection to each sensor. Bluetooth or other suitable wireless protocols may be used to communicate with the programmer instrument, which can include applications running on a tablet computer or smartphone. In use, information from the sensors may be obtained prior to implanting the trial system lead by placing the outer circle sensor portion of the patch on the patient (without also providing the inner circle portion.) The patient wears the set of sensors for a few days to obtain a baseline of activity and pain. The patient then receives the SCS trial system and measurements from the sensors continue to determine if an appreciable change in pain can be detected. In addition, as explained above, sensor measurements can be used to titrate therapy. The settings that best reduce pain in presence and absence of activity are preferably chosen at the end of the trial period.

In the example of FIG. 7, the patch includes a pair of ECG electrodes 604 and 605 for sensing electrical signals on the surface of the skin emanating from the heart from which the patient ECG can be derived or obtained. A GSR sensor 606 includes various electrodes 608 for sensing signals on the surface of the skin from which GSR can be derived or obtained. A pulse oximeter 610 includes various sensors 612 for sensing optical or other signals through the surface of the skin from which BP can be derived or obtained. An accelerometer 614 is also shown, along with a central electronics portion 616 of the trial device that includes the pulse controller, battery, etc., as well as the connector for connecting the percutaneous SCS lead (not shown.) Various interconnection lines may be provided (not shown) for connecting the various sensors of the device to the central electronics. Other components, such as the adhesive used to affix the patch to patient skin, are also not shown in this particular figure. It should be understood that in practical implementations sufficient space should be maintained around the perimeter of the patch to accommodate the adhesive for securely affixing the patch to patient skin and to keep the electrical components free of water during, for example, bathing. Hence, rather than position the sensor electrodes near the perimeter of the patch as shown in FIG. 7, a greater amount of space may be left between the various sensors and the perimeter of the patch. Wireless components and techniques may be employed, where appropriate, to relay signals between the various sensors of the patch. Still further, in at least some examples, the stimulation lead is not physically coupled to the electronics of the patch but may receive power from the electronics of the patch via, for example, electromagnetic induction.

Further information regarding an exemplary neurostimulation patch configuration that can be adapted for use with sensors is provided in U.S. patent application Ser. No. 13/938,828, filed Jul. 10, 2013, entitled “Neurostimulation Patch.”

FIG. 8 illustrates a simplified example of an embodiment of a neurostimulation patch 700 equipped for pain detection, in which a GSR sensor 701 and accelerometer 703 are shown. A pulse oximeter and an ECG sensor may also be provided but are not shown in this particular drawing. In this example, neurostimulation patch 700 is attached to the skin S of a patient and configured to deliver neurostimulation to the spinal cord SC of the patient via a percutaneous lead 704. For purposes of illustration, patch 700 and the spine of the patient are shown in cross-sectional view in FIG. 8. In this example, patch 700 includes a body member 706, a neurostimulation circuit 708 located within the body member 706, and a first connector or coupler 710 located within the body member. Among other functions, neurostimulation circuit 708 generates neurostimulation pulses for delivery to lead 704 via connector 710. The neurostimulation circuit of this example also receives signals from GSR sensor 701 via a connection line 711 and from accelerometer 703 via a connection line 713 (and optionally from other sensors not shown via other connection lines) for use in assessing patient pain, as already described.

Body member 706 includes a central portion 712 and a peripheral portion 714. In a typical implementation, central portion 712 embodies most of the circuitry (e.g., the neurostimulation circuit 708 and the connector 710) of patch 700 and serves to protect the puncture site where lead 704 passes through skin S, while the peripheral portion 714 is to affix the patch 700 to the skin S and provide a seal and, as shown, provide space for the aforementioned sensors. However, the various components may be distributed in other ways and the various portions of the patch may serve different functions in other embodiments of the neurostimulation patch. The bottom, inner or “skin side” portion (i.e. the left side in FIG. 8) of body member 706 defines an opening 716 (delineated by the dashed lines) for passage of lead 704. Opening 716 also serves to protect the puncture site since member 706 does not necessarily lie directly on the skin at the puncture site in the area of opening 716 (e.g., the opening provides a space to enable use of a gauze material over the puncture site as discussed below and also preferably provide space for coiling excess portions of the lead.) FIG. 8 shows only one opening 716 but multiple openings can be provided to accommodate passage of multiple leads into the patch for connection to circuitry 708. This allows for covering additional sites along the spinal cord to increase coverage of possible pain relieving tracts along the spinal cord.

In some embodiments, body member 706 is constructed of a flexible (e.g., pliable) material. Through the use of such a material, patch 700 may readily conform to the contours of the patient's skin, even when the skin is subjected to movement during patient activity. Accordingly, patch 700 is preferably configured to be relatively comfortable for the patient to wear. Upon implant of lead 704, patch 700 is bonded to the patient's skin, upon application of pressure. Other fixation techniques may be used to attach a neurostimulation patch to a patient in other embodiments. Examples of materials from which body member 706 may be constructed include one or more of: flexible molded polymer, silicone, polyurethane, soft poly vinyl chloride (PVC) or butyl rubber. Note that openings may be provided within the inner skin-side portion 718 of the patch to accommodate the various sensors so that those sensors may be disposed or positioned directly against the skin of the patient, if needed. For example, openings may be provided within portion 718 of the patch so that the electrodes of the GSR sensor and the ECG sensor can press against patient skin. Likewise, an opening may be provided so that optical sensors used by the pulse oximeter can beam light directly into patient skin for obtaining measurements. In some embodiments, a portion of the skin side of the patch includes a conductive polymer to provide at least one surface electrode that contacts the skin S of the patient. The surface electrode may be formed of a metallic foil or screen coated with a conductive adhesive. This electrode can be used for sensing electrical signals for use by one or more of the sensors, such as for sensing signals to obtain the surface ECG, or for other purposes.

In some embodiments, patch 700 includes or is combined with absorbing material gauze (e.g., a bandage) for absorbing blood and other body fluids. For example, a gauze material may be located over opening 716 to protect the puncture site. The gauze material could have antibacterial qualities. Alternatively, patch 700 could include circuitry to deliver an electric field that prevents formation of a biofilm and thus prevents infection. In some embodiments, the skin side of peripheral portion 714 includes a seal around the puncture site and/or around patch 700. Such a seal may protect the puncture site from infection and/or protect the components of patch 700. Preferably, the seal is waterproof to provide protection from water (e.g., to enable the patient to bathe or shower). In some embodiments, the electronics of patch 700 are waterproofed by encasing them in a water-repellent material.

The patch can be disposable or reusable. Also, in some embodiments, the electronics in patch 700 are removable to enable the patch to be changed and/or the electronics replaced. In the former case, the electronics would be detached from an old patch and then reattached to a new patch. In this manner, the patch could be changed every day or as needed. In the latter case, the electronics may be replaced or renewed (e.g., a battery recharged or replaced). In the example of FIG. 8, lead 704 may be permanently, releasably or detachably connected to patch 700 via connector 710. As an example of a permanent connection, connector 710 may include a set of conductors (e.g., contacts or other types of conductors) to which a comparable set of conductors on lead 704 are electrically coupled while providing a substantially permanent (i.e., not readily removable) fixture. For example, the lead conductors may be soldered to contacts of connector 710. As an example of a releasable connection, connector 710 may include a releasable connector that includes contacts, whereby the releasable connector is configured to accept a complementary connector (e.g., a set of contacts) on lead 704. In such a case, lead 704 may be readily connected to or disconnected from the patch 700 to, for example, facilitate implanting lead 704, changing patch 700, or changing the electronics of patch 700.

In some examples, the patch, or portions thereof, are waterproof or water resistant. The adhesive used to adhere the patch to patient skin (e.g. applied along the inner skin-side portion 718 of the patch) may incorporate a topical anesthetic (such as Lidocaine), a Steroid (such as cortisone), and/or an antihistamine (such as Benadryl™) Such compounds may be particularly advantageous to address skin allergies, skin irritation, etc., particularly for use with longer term trials.

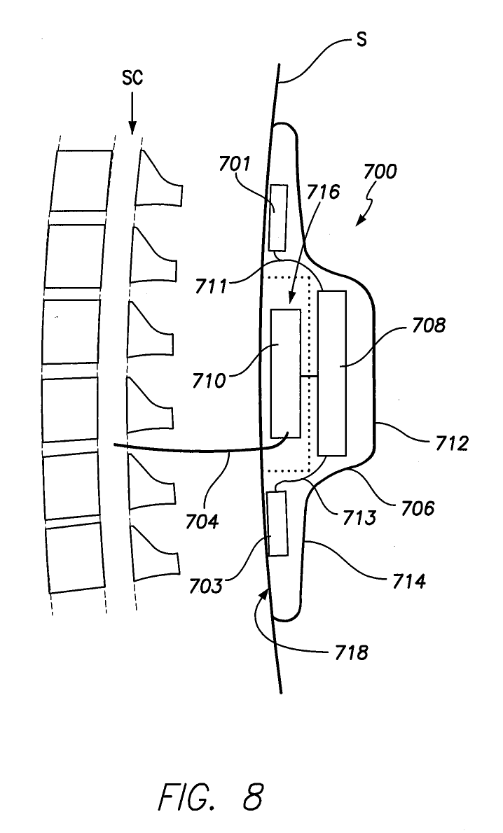

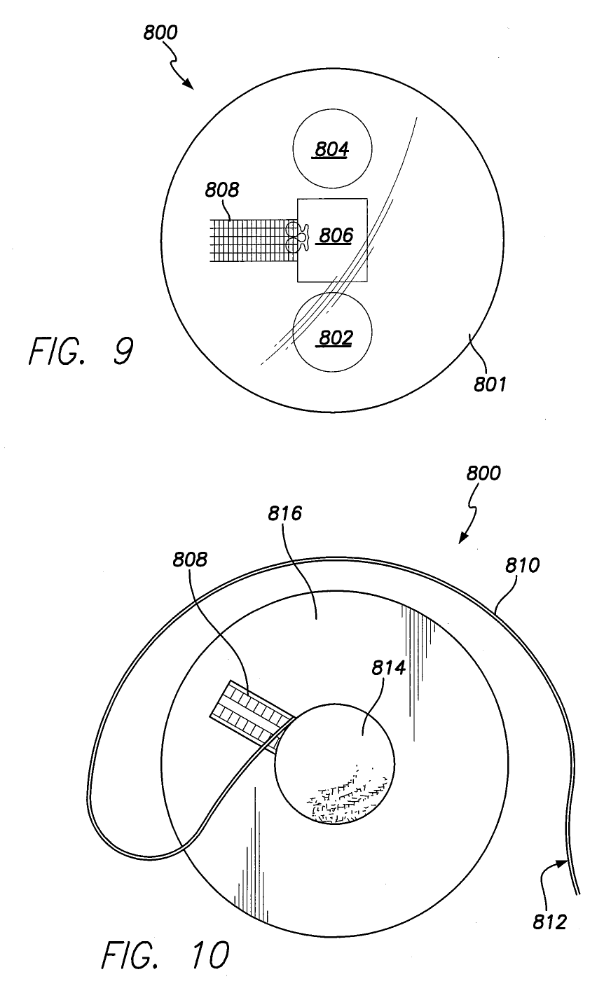

Turning now to FIGS. 9-12, illustrative patch embodiments will be briefly described by way of a couple of examples. Note that these figures do not specifically show the sensors used to provide signals to assess pain (e.g. the GSR sensor, etc.) but are nevertheless helpful in illustrating patches in which such sensors can be installed. Also, it should be appreciated that in some embodiments no sensors are provided. The patch instead includes the trial neurostimulation components but no sensors. Beginning with FIG. 9, the front, outer or top side of a patch 800 is shown, which includes a surface or pouch formed of a soft and pliable material. In the example, the patch itself is round and at least some exterior portions of the patch have a soft silicon rubber foundation. The interior of the patch includes a pair of batteries 802 and 804 and an electronics module 806, which includes various circuit components and the main connector for connecting the percutaneous lead. Other connectors 808 are also shown, as may be needed to receive connections from the various sensors (not shown.) In some examples, the percutaneous lead might be connected instead into connector 808 with the gauze extending over that component as well. FIG. 10 shows the skin-side or bottom portion of patch 800. A percutaneous lead 810 is shown with a set of eight electrodes 812 at its distal end. As can be seen, in this example, a proximal end of the lead is connected into a central portion of the patch (and particularly into electronic module 806 shown in FIG. 9.) A gauze material 814 is provided for mounting over or near the puncture site to absorb blood or other fluids. A peelable adhesive protector 816 is also shown that is adapted to be peeled away from the patch to expose adhesives for affixing and sealing the patch to the skin of the patient, after the lead has been implanted.

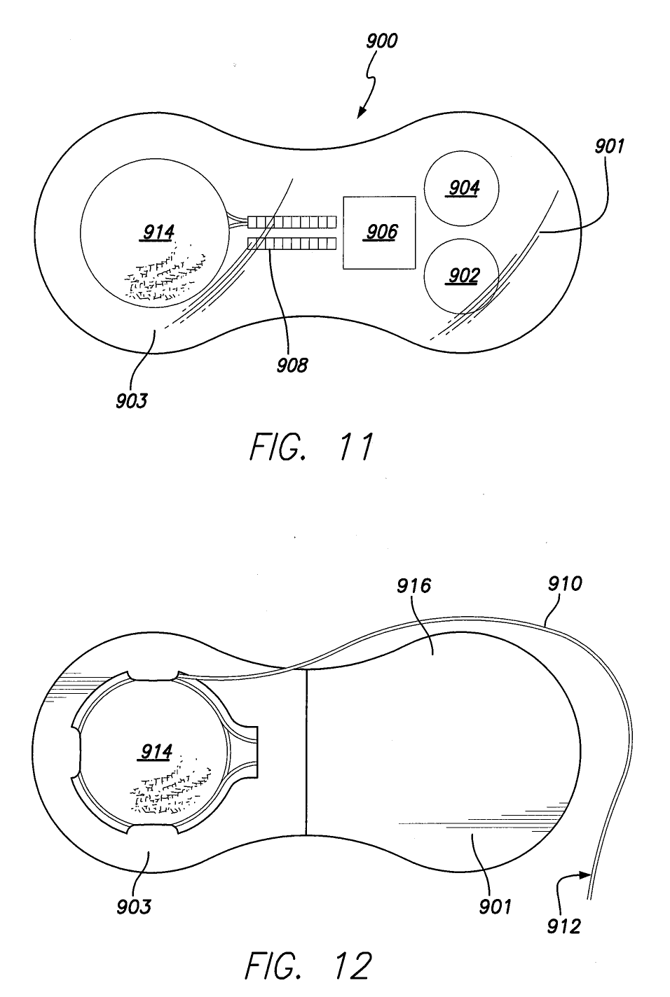

FIGS. 11-12 show an alternative embodiment where the patch is elongated. More specifically, in FIG. 11, the front, outer or top side of a patch 900 is shown, which includes an elongated surface or pouch formed of a soft and pliable material (as with the preceding example) but is elongated into a generally oval shape having first and second circular portions for installing different components of the patch. In this example, a first circular end portion 901 of the patch includes a pair of batteries 902 and 904 and an electronics module 906 including various circuit components. A second circular end portion 903 includes components for connecting the percutaneous lead, which are obscured in the figure by gauze 914. Other connectors 908 are installed within a middle portion of the patch and may be employed, e.g., to receive connections from the various sensors (not shown) or to electrically couple or connect the components of the two ends of the elongated patch together. In some examples, the proximal end of the lead is connected directly into connector 908. FIG. 12 shows the skin-side or bottom portion of patch 900. A percutaneous lead 910 is shown with a set of eight electrodes 912 at its distal end. As can be seen, in this example, a proximal end of the lead is connected into end portion 903 of the patch where it is wrapped or coiled to “take up” extra length of the lead. Gauze material 914 is provided for mounting over or near the puncture site to absorb blood or other fluids. A peelable adhesive protector 916 is adapted to be peeled away from the patch to expose adhesives for affixing and sealing the patch to the skin of the patient, after the lead has been implanted.

The foregoing exemplary systems, methods and apparatus provide one or more of the following features or advantages: a) a trial patch having a stimulator and a bandage component that also incorporates pain detection and measurement capability; b) communication of pain indices with RF from trial patch to a programmer instrument (such as a suitably-equipped smartphone); c) pain detection with GSR, activity, PPG (blood pressure), and HR; d) pain may be objectively measured before, during and after the trial; e) useful for clinical trials; f) especially useful for paresthesia-free neuromodulation using burst, etc.; and g) algorithms or procedures are provided that incorporate different sensors in various combinations.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A method for use with a trial neurostimulation device having a neurostimulation lead for implant within a patient, the method comprising:
selectively delivering neurostimulation to the patient via the lead using the trial neurostimulation device; and
detecting an indication of patient pain using the trial neurostimulation device.

2. The method of claim 1 wherein detecting an indication of patient pain comprises measuring an intensity of patient pain.

3. The method of claim 1 wherein detecting an indication of patient pain comprises tracking changes in patient pain over an interval of time.

4. The method of claim 1 wherein the trial neurostimulation device is equipped with a galvanic skin response (GSR) sensor and wherein detecting an indication of patient pain is based on GSR.

5. The method of claim 4 wherein detecting an indication of patient pain based on GSR comprises:
detecting spikes in a GSR signal sensed by the GSR sensor; and
associating changes in a number of spikes in the GSR signal over time with changes in an intensity of patient pain.

6. The method of claim 4 wherein detecting an indication of patient pain based on GSR comprises:
detecting a frequency content of a GSR signal sensed by the GSR sensor; and
associating changes in the frequency content of the GSR signal over time with changes in an intensity of patient pain.

7. The method of claim 1 wherein the trial neurostimulation device is further equipped with an activity sensor and wherein patient pain is separately detected during periods of activity and periods of relative inactivity.

8. The method of claim 1 wherein the trial neurostimulation device is further equipped with a heart rate sensor and wherein a lack of a significant increase in heart rate is indicative of a lack of patient pain.

9. The method of claim 1 wherein the trial neurostimulation device is further equipped with a blood pressure sensor and wherein a lack of significant increase in blood pressure is indicative of a lack of patient pain.

10. The method of claim 1 further including one or more of: storing parameters representative of patient pain within a device memory and transmitting parameters representative of patient pain to an external instrument.

11. The method of claim 10 wherein the parameters representative of patient pain include one or more of: a value representing an intensity of the pain; a corresponding GSR value; a corresponding heart rate value; a corresponding blood pressure value; and a corresponding activity level value.

12. The method of claim 1 further including controlling the neurostimulation in response to the indication of patient pain.

13. The method of claim 12 wherein controlling the neurostimulation includes one or more of: activating neurostimulation; deactivating neurostimulation; and adjusting one or more control parameters.

14. The method of claim 12 wherein neurostimulation is selectively activated and deactivated while the indication of patient pain is detected to assess the efficacy of the neurostimulation.

15. The method of claim 1 wherein selectively delivering neurostimulation comprises: employing a preliminary baseline interval during which no neurostimulation is delivered and baseline pain parameters are measured; and employing a subsequent trial stimulation interval during which neurostimulation is delivered and pain parameters are detected for comparison against the baseline parameters.

16. A trial neurostimulation device having a neurostimulation lead for implant within a patient, the device comprising:
a neurostimulation system operative to deliver neurostimulation to the patient via the lead;
a pain detector operative to detect an indication of patient pain.

17. The trial neurostimulation device of claim 16 further comprising a controller operative to control at least one function of the neurostimulation device in response to the indication of patient pain.

18. A trial neurostimulation device having a neurostimulation lead for implant within a patient, the device comprising:
means for selectively delivering neurostimulation to the patient using the lead;
means for detecting an indication of patient pain; and
means for controlling at least one function of the device in response to the indication of patient pain.

19. A neurostimulation patch device for use with an implantable neurostimulation lead for implant within a patient, the patch device comprising:
a body member having a bottom portion adapted to be detachably affixed to patient skin;
a neurostimulation circuit within the body member and configured to output neurostimulation signals;
a connector located within the body member and configured to electrically couple the neurostimulation circuit to the implantable lead, wherein the bottom portion of the body member defines an opening for passage of an end of the implantable lead for connection to the connector; and
at least one sensors operative to sense physiological signals mounted within the body member.

20. The neurostimulation patch device of claim 19 further comprising a pain detection system operative of detect an indication of patient pain based on signals received from the at least one sensor.

21. The neurostimulation patch device of claim 20 wherein the at least one sensor comprises a galvanic skin response (GSR) sensor and wherein the pain detection system detects an indication of patient pain based on GSR signals.

22. The neurostimulation patch device of claim 21 wherein the at least one sensor further comprises one or more of: an electrocardiogram (ECG) sensor; a pulse oximeter; and a patient activity sensor.

23. The neurostimulation patch device of claim 20 further comprising a transmission device operative to transmit parameters associated with patient pain to an external instrument.

24. The neurostimulation patch device of claim 19 wherein the body member further comprises a central portion and a peripheral portion, the peripheral portion including a skin adhesive material.

25. The neurostimulation patch device of claim 24 wherein the skin adhesive material is formed around a perimeter of the peripheral portion for sealing the body member over an implant site of the lead.
